(19) United States
(12) Patent Application Publication
(10) Pub. No.: US 2018/0326074 A1
(43) Pub. Date: Nov. 15, 2018

(54) METHODS FOR SYNTHESIZING PEPTIDE-TAGGED PEGYLATED CHITOSAN

(71) Applicant: NANORA PHARMA INC., Montreal (CA)

(72) Inventors: Meenakshi MALHOTRA, Montreal (CA); Satya PRAKASH, Brossard (CA)

(21) Appl. No.: 15/775,552

(22) PCT Filed: Nov. 8, 2016
(86) PCT No.: PCT/CA2016/051299

§ 371 Date: May 11, 2018

Related U.S. Application Data
(60) Provisional application No. 62/254,059, filed on Nov. 11, 2015.

(51) Int. Cl. A61K 47/36; A61K 47/64; A61K 47/69; C07K 14/005; C07K 1/113; C08B 37/08; C08G 65/332; C08G 81/00; C09D 163/00; C08K 9/04

(52) U.S. Cl. CPC A61K 47/36; A61K 47/645; A61K 47/6939; C07K 14/005; C07K 1/113; C12N 2740/16322; C08G 65/332; C08G 81/00; C09D 163/00; C08K 9/04; C08B 37/003

(57) ABSTRACT

Provided are synthetic schemes for the synthesis of a derivatized chitosan polymer, grafted with polyethylene glycol (PEG) and a peptide such as a cell-targeting/cell penetrating peptide. In alternative embodiments, provided are synthetic schemes for the preparation of a peptide tagged PEGylated phthaloyl chitosan (CS-O-PEG-peptide), or a CS-O-PEG-TAT if the peptide is a TAT. Provided are synthetic schemes for the preparation of a PEGylated Phthaloyl Chitosan (CS-PH-O-PEG-peptide), or a CS-PH-O-PEG-TAT if the peptide is a TAT. In alternative embodiments, provided are protocols and synthetic schemes for the preparation of a phthaloyl chitosan and a homo-functional di-carboxylic acid polyethylene glycol (COOH-PEG-COOH). Provided are protocols and synthetic schemes for the preparation of a peptide tagged PEGylated chitosan (CS-O-PEG-peptide). Provided are peptide-tagged PEGylated chitosan (CS-O-PEG-peptide) (optionally CS-O-PEG-TAT if the peptide is a TAT) made by a method or synthetic scheme as provided herein.

### FIELD OF THE TECHNOLOGY

The invention generally relates to synthetic and medicinal chemistry. In alternative embodiments, provided are protocols and synthetic schemes for the synthesis of a derivatized chitosan polymer, grafted with polyethylene glycol (PEG) and a peptide such as a cell-targeting/cell penetrating peptide.

### BACKGROUND

Chitosan is linear polysaccharide composed of β-(1-4)-linked D-glucosamine (deacetylated unit) and N-acetyl-D-glucosamine (acetylated unit) and is biocompatible and biodegradable in nature. It is commercially produced by the deacetylation of chitin (obtained from the exoskeleton of crabs and shrimps). Commercially available chitosan polymer is about 75 to 85% deacetylated and can be used for biomedical purposes. It is available in low, medium and high molecular weights that spans a molecular weight range of 10 to 220 KDa.

The presence of anime groups on chitosan has a Pka of approximately 6.5, which protonate at an acidic pH. Chitosan is only soluble in 1-2% acetic acid solution and is insoluble in water at neutral pH. The preserve of amine groups in chitosan makes it a desirable candidate for biomedical applications such as gene delivery, wherein the amine (positive charges) on the chitosan electrostatically bind with the negative charges on a nucleic acid, for example, a DNA or a gene, to form a polyplex, called a polymeric nanoparticle.

### SUMMARY

In alternative embodiments, provided are methods and synthetic schemes for the preparation of a phthaloyl chitosan (CSPH) comprising;

(a) providing a chitosan, wherein optionally the chitosan has a range of molecular weight (MW) of between about 10-220 KDa, and optionally the chitosan is substantially pure with a deacetylation of between about 75 to 85%

(b) providing a solution of phthalic anhydride, optionally at about 465.90 millimolar (mM), or equivalent, in a solvent comprising an N,N-Dimethyl Formamide (DMF) or equivalent;

(c) mixing a sufficient amount of the chitosan of (a) with the solution of (b) such that the final amount of phthalic anhydride or equivalent is in 3 mole excess of the chitosan;

(d) stirring the mixture of (c) under a nitrogen atmosphere or equivalent non-oxygen atmosphere at a temperature above a 100° C., optionally stirring for between about 6 to 10 hours, or for 6, 7, 8, 9, or 10 hours, and optionally stirring at a temperature of between about 100° C. and 120° C., or at 110° C.;

(e) cooling the stirred mixture first to about room temperature (RT) or between about 21° C. to 24° C., adding the reaction mix in excess of water at 0° C. to 15° C. (optionally cooling in ice water or ice cold water) to generate a precipitate of phthaloyl chitosan (CSPH);

(f) isolating the precipitate of CSPH, optionally by filtering, and washing the CSPH with a solvent comprising a methanol or equivalent, in excess, optionally washing overnight or between about 12 to 16 hours; and

(g) drying, optionally vacuum drying, the CSPH-comprising solvent to yield a CSPH product.

In alternative embodiments, provided are methods and synthetic schemes for the preparation of a carboxyl terminated PEG-monomethyl ether (mPEG-COOH), comprising;

(a) providing a polyethylene glycol (PEG)-monomethyl ether (PEG-MME), wherein optionally the PEG-MME has a range of molecular weight (MW) of between about 2000 Da to 10,000 Da, and optionally the PEG-MME is substantially pure;

(b) providing a solvent comprising a toluene or equivalent;

(c) mixing the PEG-MME with the solvent of(b) to a range of between about 10% to 30% PEG-MME, under a nitrogen, atmosphere or equivalent non-oxygen atmosphere at between about 50° C. and 70° C. or at 60° C.;

(d) providing a solution of succinic anhydride in pyridine at concentration equivalent to about 12 grams (gms) succinic anhydride dissolved in between about 50 ml to 100 ml pyridine;

(e) adding the solution of (d) dropwise or incrementally to the PEG-MME solution of step (c) until succinic anhydride is at about a 4-fold mole excess of the PEG-MME;

(e) stirring with refluxing the final solution of (e) at a temperature above 100° C., or at a temperature between about 100° C. to 120° C, or 110° C., optionally for between about 5 to 15 hours, or 6 to 12 hours;

(f) cooling the stirred solution of (e) to about room temperature (RT) or between about 21° C. to 24° C., and precipitated with a solvent comprising an ethyl ether (EE), optionally using about 2 liters (L) EE or equivalent;

(g) isolating the precipitate of (f), optionally by filtering, and re-dissolving fee precipitate by adding a solvent comprising a chloroform or equivalent and again re-precipitating in diethyl ether or equivalent, optionally the solvent, comprising the equivalent of 100 ml chloroform or equivalent and 2 L diethyl ether or equivalent; and

(h) drying, optionally vacuum drying, the solvent of (g) to yield a dry carboxyl terminated PEG-monomethyl ether (mPEG-COOH) product.

In alternative embodiments, provided are methods and synthetic schemes for the preparation of a PEG-grafted phthaloyl chitosan polymer (CSPH-O-mPEG), comprising;

(a) providing a phthaloyl chitosan (CSPH), optionally a phthaloyl chitosan (CSPH) made by the method as provided herein, and dissolving the CSPH in a solvent comprising a pyridine or equivalent, stirring (optionally stirring overnight, or between about 5 to 20 hours or 12 to 16 hours) at about room temperature (RT) or at between about 21° C., to 24° C.;

(b) providing a carboxyl terminated PEG-monomethyl ether (mPEG-COOH), or the carboxyl terminated PEG-monomethyl ether (mPEG-COOH) product as provided herein, and dissolving in a solvent comprising a toluene or equivalent at between about 50° C. and 70° C., or about 60° C., under a nitrogen atmosphere or equivalent non-oxygen atmosphere, and after complete dissolution in the solvent add a thionyl chloride (SOCl₂) at an amount of between about equimolar to 2-fold molar excess;

(c) stirring and refluxing under boiling conditions the final solution of (b) for between about 5 to 10 hours (hrs) or 6 to 8 hrs, followed by degassing to remove excess SO₂ and thioyl chloride, to generate a mPEG-COCl product;

(d) lowering the temperature of the mPEG-COCl product-comprising solution of (c) to about room temperature (RT) or at between about 21° C. to 24° C., and adding dropwise or incrementally the CSPH solution of step (a), optionally adding about 200 ml, and stirring at about room temperature (RT) or at between about 21° C. to 24° C., for between about 1 to 5 hrs or about 2 hrs under a nitrogen atmosphere or equivalent non-oxygen atmosphere;

(e) stirring the final solution of (d) for between about 18 hrs to 30 hrs or about 24 hrs under boiling conditions or conditions comprising boiling and refluxing; and

(f) precipitating a CSPH-O-mPEG product in excess of a solvent comprising a methanol or equivalent, thereby generating a CSPH-O-mPEG product, and optionally drying (optionally vacuum drying) to generate a dry mPEG-PHCS or CSPH-O-mPEG product.

In alternative embodiments, provided are methods and synthetic schemes for the preparation of a hydroxyl terminated PEGylated Phthaloyl Chitosan (CSPH-O-PEG-OH), comprising;

(a) mixing a CSPH-O-mPEG, optionally made by the method for making a CSPH-O-mPEG as provided herein, with an about equimolar amount of Aluminum Chloride, using ethanethiol or equivalent as a solvent, and stirring (optionally for between, about 10 to 30 hrs, or 12 to 24 hrs) at about room temperature (RT) or at between about 21° C. to 24° C.; and

(b) diluting the reaction mix with water (optionally a two-fold dilution), and acidified (optionally with HCl or 10% HCl, optionally to about pH 1, 2 or 2.5, or between about pH 0.5 and 2.5) to form a precipitate, and vacuum, filtering the precipitate to generate a CSPH-O-PEG-OH product, and optionally further purifying by extraction in a solvent comprising a dichloromethane (DCM) or equivalent (optionally extracting 3 times in DCM).

In alternative embodiments, provided are methods and synthetic schemes for the preparation of a carboxyl terminated PEGylated Phthaloyl Chitosan (CSPH-O-PEG-COOH), comprising;

(a) mixing a CSPH-O-PEG-COOH, optionally a CSPH-O-PEG-COOH made by a method as provided herein, with a succinic anhydride in a solvent comprising a toluene or equivalent, wherein the succinic anhydride is added until it is in 4 mole excess of the CSPH-O-PEG-COOH, and the succinic anhydride is added dropwise or incrementally as a predissoived succinic anhydride in a solvent comprising a pyridine or equivalent;

(b) stirring the mixture of (a) at about 100° C. for between about 8 to 16 hours, or about 12 hrs, under a nitrogen atmosphere or equivalent non-oxygen atmosphere, and lowering the temperature of the mixture to about room temperature (RT) or between about 21° C. to 24° C., and

(c) precipitating the CSPH-O-PEG-COOH product from the mixture of (b) by adding a solvent comprising a methanol or equivalent in excess, thereby generating a CSPH-O-PEG-COOH product, and optionally further comprising drying, or vacuum drying to generate a dry CSPH-O-PEG-COOH product.

In alternative embodiments, provided are methods and synthetic schemes she preparation of a PEGylated Phthaloyl Chitosan conjugated to a peptide (CS-PH-O-PEG-peptide) having an amine group (optionally a terminal amine group), optionally the peptide comprising a trans-activating transcriptional activator (TAT) peptide (CS-PH-O-PEG-TAT), comprising:

(a) dissolving (or mixing) a CSPH-O-PEG-COOH optionally a CSPH-O-PEG-COOH made by a method as provided herein, in a solvent comprising a N,N-Dimethyl Formamide (DMF) or equivalent, optionally at between about 50° C. to 70° C. or at about 60° C. for between about 0.5 to 5 hours or for about 2 hrs.

(b) adding a solution of ethylene dichloride (1,2 dichoroethane, or EDC) predissoived in DMF, wherein EDC is in 1.3 mole equivalents of the CSPH-O-PEG-COOH polymer (in alternative embodiments, the concentration is not so important, and an approximate amount of DMF can be taken, e.g. enough to dissolve EDC in it as it dissolves very readily) in a dropwise or incrementally to the mixture of (a), and then the peptide pre-dissolved (in alternative embodiments, the concentration for pre-dissolving is not so important; it can be an approximate volume taken to easily dissolve the peptide in DMF before adding it into the reaction mix) in a solvent comprising DMF or equivalent is added dropwise or incrementally, wherein the amount of peptide used is its equimoles of the CSPH-O-PEG-COOH polymer;

(c) adding (or mixing in) a 4-dimethylaminopyridine (DMAP) pre-dissolved in a solvent comprising a DMF at the equivalent of 0.1 moles of the CSPH-O-PEG-COOH polymer and then stirring the solution, optionally for between about 12 to 36 hrs or for about 24 hrs, optionally at between about 25° C. to 60° C. or at about 40° C.;

(c) lowering the temperature of the mixture of (b) to about room temperature (RT) or between about 21° C. to 24° C., and precipitated in excess water (optionally 1 L water), and a CSPH-O-PEG-peptide precipitate (optionally a CSPH-O-PEG-TAT) isolated, optionally by vacuum filtering; and

(d) washing the isolated product of (e) (optionally a filtered product), optionally in a solvent comprising a methanol or equivalent to yield a CSPR-O-PEG-peptide product (optionally a CSPH-O-PEG-TAT product), and optionally further comprising drying (optionally filter drying) to produce a dry CSPH-O-PEG-peptide (optionally a CSPH-O-PEG-TAT product).

In alternative embodiments, provided are methods and synthetic schemes the preparation of a peptide tagged PEGylated chitosan (CS-O-PEG-peptide) (optionally CS-O-PEG-TAT if the peptide is TAT), comprising:

(a) dissolving or mixing a (CS-O-PEG-peptide) (optionally CS-O-PEG-TAT if the peptide is TAT), optionally the CS-O-PEG-peptide made by a method as provided herein, in a solvent comprising a DMF or equivalent to an amount equivalent to 5 grams (gm) of CS-O-PEG-peptide in 150 ml DMF, wherein the dissolving it at a temperature of between about 80° C. to 100° C. or 70° C. to 110°0 C.;

(b) adding dropwise or incrementally to the mixture of (a) a hydrazine monohydrate (HMH) (optionally about 64-65% HMH) (optionally 13 mL of HMH, or HMH is used in 1:7.6 v/v ratio to DMF)), and stirring at between about 80° C. to 100° C. or 70° C. to 110° C. under a nitrogen atmosphere or equivalent non-oxygen atmosphere, optionally for between about 0.5 to 4 hrs or for about 2 hrs; and

(c) lowering the temperature of the mixture of (b) to about room temperature (RT) or between about 21° C. to 24° C., and precipitating in a solvent comprising an ethanol or equivalent, thereby producing a peptide tagged PEGylated chitosan (CS-O-PEG-peptide) (optionally CS-O-PEG-TAT if the peptide is a TAT) product,

and optionally the precipitate is washed in a solvent comprising an ethanol or equivalent optionally for about 10 to 20 hrs of for about 12 to 16 hrs and isolated (optionally isolated by filtering, optionally by vacuum filtering), and the isolated precipitate is dried (optionally air dried), to produce a dry peptide tagged PEGylated chitosan (CS-O-PEG-peptide) (optionally CS-O-PEG-TAT if the peptide is a TAT) product.

In alternative embodiments, provided are methods and synthetic schemes the preparation of a peptide tagged PEGylated chitosan (CS-O-PEG-peptide) (optionally CS-O-PEG-TAT if fee peptide is TAT), comprising the methods or synthetic schemes as provided herein.

In alternative embodiments, provided are peptides tagged PEGylated chitosan (CS-O-PEG-peptide) (optionally CS-O-PEG-TAT if the peptide is a TAT) made by the methods or synthetic schemes as provided herein (optionally about 60 to 100% pure),

wherein optionally the CS-O-PEG-peptide is: (a) soluble in 0.5% to 1% acetic acid; (b) has 6 times higher siRNA binding efficiency than a CS-O-PEG-peptide of less purity; (c) formed nanoparticles in the range of between about 200 nm to 250 nm with a surface charge (zeta potential) of 15-20 mV; or (d) any combination of (a), (b) and (c).

In alternative embodiments, provided are methods and synthetic schemes for the preparation of a phthaloyl chitosan and a homobifunctional di-carboxylic acid polyethylene glycol (COOH-PEG-COOH) comprising:

(a) providing a chitosan, wherein optionally the chitosan has a range of molecular weight (MW) of between about 10 to 220 KDa, and optionally the chitosan is substantially pure, optionally with about 75 to 85% deacetylation;

(b) providing a solution of phthalic anhydride or equivalent, optionally equivalent to 465.90 millimolar (mM), in a solvent comprising an N,N-Dimethyl Formamide (DMF) or equivalent;

(c) mixing a sufficient amount of the chitosan of (a) with the solution of (b) such that the final amount of phthalic anhydride or equivalent is in 3 mole excess of the chitosan;

(d) stirring the mixture of (c) under a nitrogen atmosphere or equivalent non-oxygen atmosphere at a temperature above 100° C. or between 100° C. and 120° C. or at about 100° C., optionally stirring for between about 6 to 10 hours, or for 6, 7, 8, 9, or 10 hours;

(e) cooling the stirred mixture first to about room temperature (RT) or between about 21° C. to 24° C. then adding the reaction mix in excess of water at 0° C. to 15° C. (optionally cooling in ice water or ice cold water) to generate a precipitate of phthaloyl chitosan (CSPH);

(f) isolating the precipitate of CSPH, optionally by filtering, and optionally further comprising washing the CSPH with a solvent comprising a methanol or equivalent, in excess, optionally washing overnight or between about 12 to 16 hours;

(g) drying, optionally vacuum drying, the CSPH-comprising solvent to yield a CSPH product;

(h) providing a homo-bifunctional dihydroxy-polyethylene glycol (OH-PEG-OH), optionally at between about 2000 Da to 10,000 Da), and dissolving in a solvent comprising a toluene or equivalent to an about 10% to 30% v/v OH-PEG-OH solution at a temperature of between about 40° C. to or at about 60° C. under a nitrogen atmosphere or equivalent non-oxygen atmosphere;

(i) adding or mixing into the mixture of (h) a succinic anhydride to a 4 mole excess of OH-PEG-OH, wherein the succinic anhydride is dissolved in a solvent comprising a pyridine or equivalent (optionally at an equivalent of 12 grams of succinic anhydride dissolved in about 50 ml to 100 ml of pyridine) and added in a dropwise or incrementally, and then stirring and refluxing for between about 1 to 15 hrs or about 6 to 12 hrs at a temperatures of between about 100° C. to 110° C.; and

(j) cooling the mixture of (i) to about room temperature (RT) or to between about 21° C. to 24° C. and precipitating a COOH-PEG-COOH with a solvent comprising an ethyl ether or equivalent (optionally 2 L of ethyl ether), thereby generating a COOH-PEG-COOH product,

Optionally further comprising isolating the precipitated COOH-PEG-COOH product (optionally by filtering), and optionally redissolving the precipitate with chloroform (optionally using 100 ml) and precipitating again with diethyl ether (optionally using 2 L) (first dissolving in chloroform and then precipitating in diethyl ether), and optionally drying (optionally vacuum drying) to generate a dried COOH-PEG-COOH product.

In alternative embodiments, provided are methods and synthetic schemes for the preparation of a COOH-PEG-COCl, comprising:

(a) providing a COOH-PEG-COOH, or a COOH-PEG-COOH made by a method as provided herein, and dissolving the COOH-PEG-COOH in a solvent comprising a toluene or equivalent (optionally at an equivalent of 330 grams COOH-PEG-COOH in 600 ml toluene or equivalent) at a temperature of between about 40° C. to 80° C. or at about 60° C. under a nitrogen atmosphere or equivalent non-oxygen atmosphere to effect complete dissolution of the COOH-PEG-COOH in the toluene or equivalent; and

(b) adding an equimolar or about a 2 fold molar excess of a thionyl chloride (SOCl₂) to the COOH-PEG-COOH-comprising mixture of (a), and stirring or mixing the mixture at 100° C. or refluxing under boiling conditions for between about 2 to 10 hrs or 6 to 8 hrs, followed by degassing to remove excess SO₂ gas and thioyl chloride (in alternative embodiments, the term “excess” means any residual gas that is produced during the reaction is purged out of the reaction mixture under nitrogen atmosphere or inert conditions) to generate a COOH-PEG-COCl product.

In alternative embodiments, provided are methods and synthetic schemes for the preparation of a polyethylene (PEG)-grafted phthaloyl chitosan polymer (CSPH-O-PEG-COOH or COOH-PEG-PHCS), comprising:

(a) providing a COCl-PEG-COCl, optionally a COCl-PEG-COCl made by a method as provided herein;

(b) providing a phthaloyl chitosan (CSPH, or PHCS), optionally a phthaloyl chitosan (CSPH) made by a method as provided herein, and dissolving the CSPH in a solvent comprising a pyridine or equivalent (optionally in 200 ml of pyridine or equivalent), stirring (optionally stirring overnight, or between about 5 to 20 hours or 12 to 16 hours) at about room temperature (RT) or at between about 21° C. to 24° C.;

(c) adding the CSPH-comprising pyridine or equivalent solution of step (b) drop-wise or incrementally to the COCl-PEG-COCl of step (a) to an amount of between about 5 to 10 times mole excess CSPH (as compared to COCl-PEG-COCl) and stirring or mixing at about room temperature (RT) or at between about 21° C. to 24° C. under a nitrogen atmosphere or equivalent non-oxygen atmosphere, optionally stirring or mixing for between about 1 to 4 hrs or for about 2 hrs, followed by stirring or mixing (optionally for between about 12 to 30 hours or for about 24 hrs) at about 100° C., optionally refluxed at 100° C. or to boil; and

(d) precipitating a COOH-PEG-PHCS product in 1 to 2 L of water at room temperature (RT) or at between about 21° C. to 24° C., and optionally drying (optionally vacuum drying) to produce a dry COOH-PEG-PHCS product.

In alternative embodiments, provided are methods and synthetic schemes for the preparation of a PEGylated Phthaloyl Chitosan (CS-PH-O-PEG-peptide) (or CS-PH-O-PEG-TAT if the peptide is a TAT), comprising:

(a) providing a COOH-PEG-PHCS (or CSPH-O-PEG-COOH), or a CSFH-O-PEG-COOH made by a method as provided herein, and dissolving in a solvent comprising a N,N-Dimethyl Formamide (DMF) or equivalent (optionally in an amount equivalent to 5 grams of CSPH-O-PEG-COOH in 250 ml of DMF or equivalent) at a temperature of between about 40° C. to 80° C. or at about optionally for between about 1 to 3 hrs or for 2 hrs;

(b) providing an ethylene dichlorlde (1,2 dichoroethane, or EDC) pre-dissolved in a solvent comprising a DMF or equivalent (optionally in an amount equivalent to 543 mg of EDC pre-dissolved in 25 ml of DMF), and adding dropwise or incrementally to the mixture of (a), wherein EPC is in 1.3 mole equivalents of the CSPH-O-PEG-COOH polymer;

(c) providing a peptide having a terminal amine (optionally a TAT peptide) dissolved in DMF (optionally in an amount equivalent to 2.92 grams of TAT peptide (molecular weight (MW) 1338.65) pre-dissolved in 20 ml of DMF), and adding dropwise to the mixture of (b), wherein the amount of peptide used is in equirnoles of the CSPH-O-PEG-COOH polymer;

(d) providing a mixture of 4-dimethylaminopyridine (DMAP) in DMF, equivalent to 0.1 moles of the CSPH-O-PEG-COOH polymer (optionally in an amount equivalent to 26 mg of DMAP pre-dissolved in 5 ml of DMF), and adding to the mixture of (c) dropwise or incrementally, and stirred at a temperature of between about 25° C. to 60° C. or at 40° C., optionally for about 24 hrs;

(e) the mixture of (d) is lowered to about room temperature (RT) or at between about 21° C. to 24° C. and precipitated in water 1 L to generate a CS-PH-O-PEG-peptide (or CS-PH-O-PEG-TAT if the peptide is a TAT) product,

and optionally the CS-PH-O-PEG-peptide precipitate product is isolated, optionally by filtering,

and optionally further comprising washing the filtered product in methanol, and optionally filter drying.

In alternative embodiments, provided are methods and synthetic schemes for the preparation of a peptide tagged PEGylated phthaloyl chitosan (CS-O-PEG-peptide) (or CS-O-PEG-TAT if the peptide is a TAT), comprising:

(a) providing a CSPH-O-PEG-peptide, or a CSPH-O-PEG-peptide made by a method as provided herein, and dissolving or mixing in DMF (optionally equivalent to 5 grams of CSPH-O-PEG-COOH is dissolved in 100 (to 150 ml) of DMF at a temperature of between about 60° C. to 100° C., or at about 80° C.; and adding in dropwise or incrementally a hydrazine monohydrate (optionally 13 ml of hydrazine monohydrate), and stirring at a temperature of between about 60° C. to 100° C., or at about 80° C., optionally for about 2 hrs or 1 to 4 hrs, a nitrogen atmosphere or equivalent non-oxygen atmosphere, HMH is used in 1:7.6 v/v ratio to DMF; and

(b) lowering the temperature of the mixture of (a) to about room temperature (RT) or at between about 21° C. to 24° C., and precipitating a CS-O-PEG-peptide in ethanol to generate a CS-O-PEG-peptide product,

and optionally further comprising washing the precipitate product at least once again in ethanol, optionally for between about 12 to 16 hours, and optionally vacuum filtering, and optionally drying (optionally air drying) to generate a dry CS-O-PEG-peptide product.

Provided are methods and synthetic schemes for the preparation of a peptide tagged PEGylated chitosan (CS-O-PEG-peptide) (optionally CS-O-PEG-TAT if the peptide is TAT), comprising the method or synthetic scheme as set forth in FIG. 1 and/or FIG. 4.

Provided are exemplary synthetic schemes capable of making gram or kilogram quantities of peptide-tagged PEGylated chitosan polymer manufactured according to current good manufacturing practices (cGMP's), consistent with US FDA requirements for human use.

All publications, databases, patents, and patent applications cited in this specification are herein expressly incorporated by reference as if each was specifically and individually indicated to be incorporated by reference.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates a schematic representation of an exemplary synthesis route for the preparation of peptide-tagged PEGylated Chitosan polymer. This scheme utilizes a total of steps, as described in detail in the Examples below. The main (or intermediate) products formed during the synthesis, designated as A, B, C and D are characterized using FTIR analysis to confirm the presence of chemical modifications.

FIG. 2(A), FIG. 2(B), FIG. 2(C), FIG. 2(C) graphically illustrate characterization of an exemplary polymer as provided herein using FTIR.

FIG. 2(A) Phthaloyl Chitosan (PHCS): vmax/cm⁻¹ 1775, 1704 carbonyl anhydride), 1150-1000 (pyranoses, and 720 (arom). The appearance of peak 1775 and 1704 Indicate the preserve of phthaloyl group on chitosan.

FIG. 2(B) PEGylated Phthaloyl Chitosan (PHCS-PEG): vmax/cm⁻¹ 2864 (C-H stretching), 1064 (C-O stretching) of PEG, 1774, 1710 (carbonyl anhydride) and 720 (arom) of phthalimido group on chitosan.

FIG. 2(C) TAT peptide-tagged PEGylated Phthaloyl chitosan (PHCS-PEG-TAT): vmax/cm⁻¹ 2867 (C—H stretching), 1067 (C—O stretching) of PEG,, 1774, 1710 (carbonyl anhydride) and 720 (arom) of phthalimido group on chitosan, 1659 and 1550 (amides) in TAT peptide.

FIG. 2(D) Deprotected TAT peptide-tagged PEGylated chitosan (CS-PEG-TAT): vmax/cm⁻¹ 2867 (C—H stretching), 3060 (C—O stretching) of PEG, 1648 and 1543 (amides) in TAT peptide and chitosan.

The presence of peaks 1659 and 1550 in FIG. 2(C) in comparison to FIG. 2(B), indicate the conjugation of TAT peptide onto the polymer, as this peak shows the presence of amides that belong to TAT peptide. The absence of peak 1775 and 1704 in FIG. 2(D), as compared to (FIG. 2A, B and C) indicate the complete removal of phthaloyl (protecting) group from the polymer.

FIG. 3 illustrates a gel retardation assay showing siRNA binding efficiency; the gel retardation assay was performed so evaluate maximum gene (siRNA) loading efficiency in the exemplary nanoparticles as provided herein, siRNA at different weight ratios were mixed with an exemplary synthesized polymer (CS-PEG-TAT). A maximum of 12 μg of siRNA was observed to bind efficiently with the synthesized polymer (CS-PEG-TAT) dissolved at 0.5 mg/ml concentration in 0.5% acetic acid solution (pH adjusted to 5.5).

FIG. 4 illustrates a schematic representation of an exemplary synthesis route for the preparation of peptide-tagged PEGylated Chitosan polymer, The synthesis route eliminates the use of a Ethanethiol step as used in the exemplary scheme as illustrated in FIG. 1. The ethanethiol step utilizes the de-etherification reaction to get rid of the mono-methyl ether group on mPEG. However, in this exemplary scheme we are utilizing a homo-bifunctional PEG instead of mPEG. This exemplary scheme reduces the number of steps of an alternative exemplary provided herein (e.g., see FIG. 1) to a total of 5 steps (see e.g., FIG. 4).

Like reference symbols in the various drawings indicate like elements.

### DETAILED DESCRIPTION

Provided herein are reproducible, scaled-up methods to form a derivative of a chitosan polymer, grafted with a hydrophilic polymer, polyethylene glycol (PEG), either mono or bi-functional, used as a linker, to conjugate a cell-targeting or a penetrating peptide (CS-PEG-TAT). In exemplary embodiments, we have used a TAT peptide (rich in arginines). In alternative embodiments, this exemplary derivatized polymer (CS-PEG-TAT) is used to form nanoparticles with siRNA.

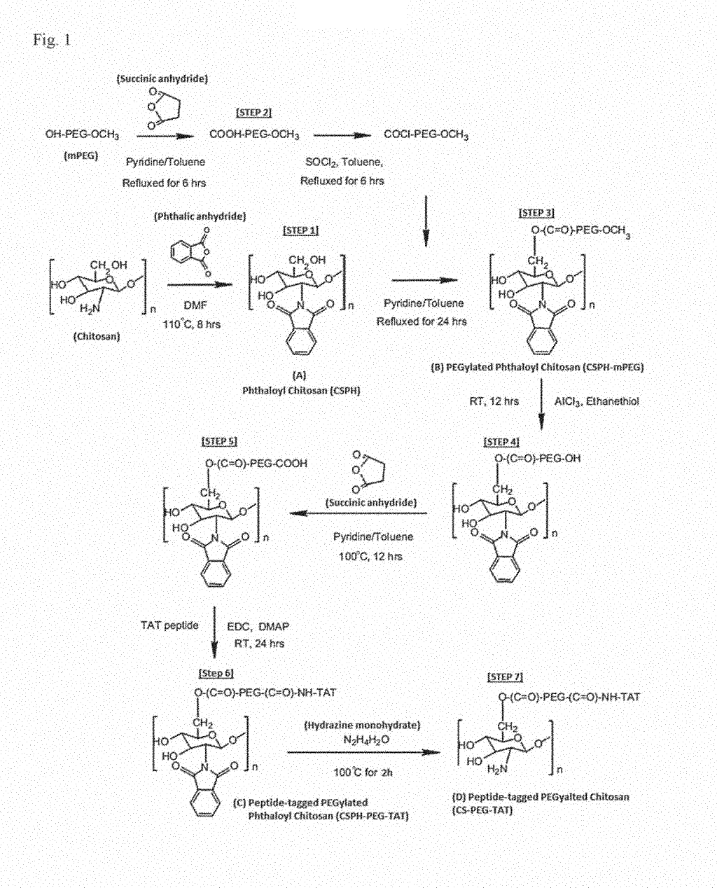

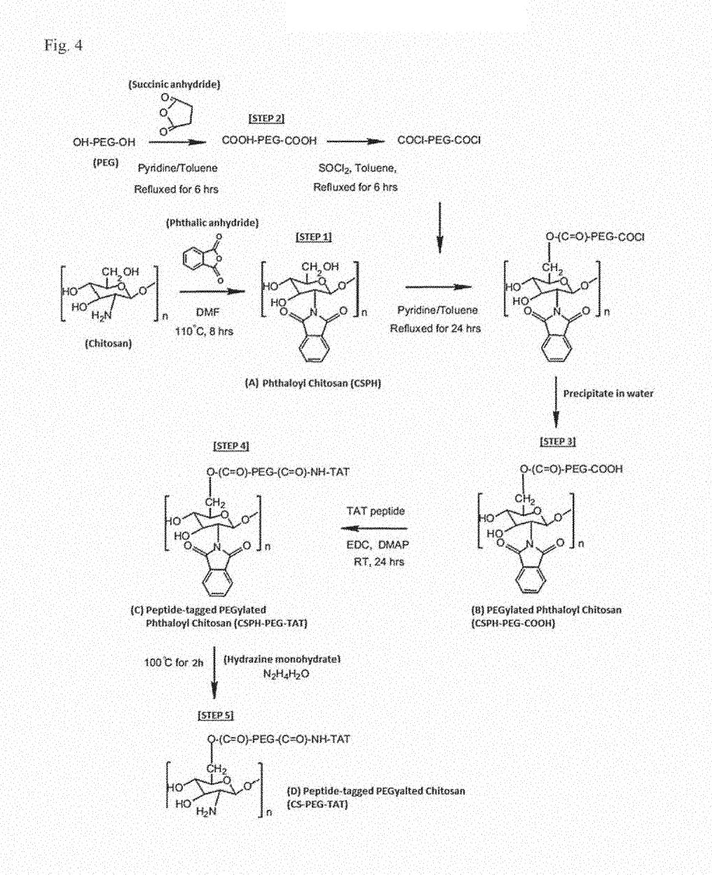

Provided herein are two exemplary scaled-up procedures for the preparation of CS-PEG-TAT polymer, which can be manufactured at an industrial scale under cGMP facilities. The first exemplary procedure is the optimized scale-up of the original method as reported previously (see e.g., Malhotra. et al. 2013a). This method has a total of synthetic steps as illustrated for example in FIG. 1. The second exemplary method is a variation of the first method, wherein the use of a mono-functional PEG (first method) is replaced with a homo-bifunctional PEG (proposed in second method), which leads to reduction of the total synthesis steps from 7 to 5, as illustrated in FIG. 4.

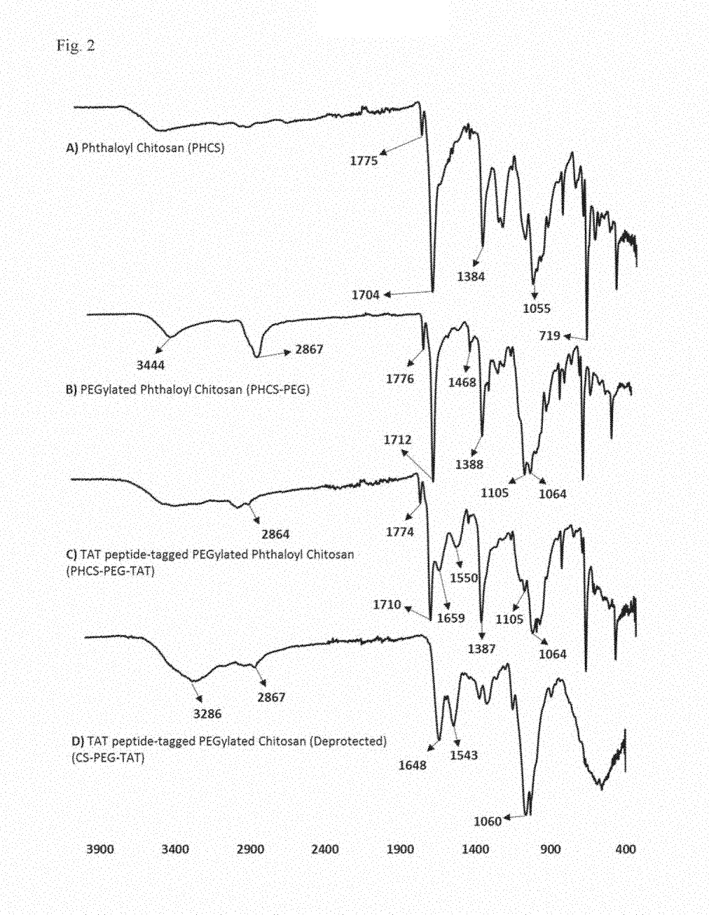

In alternative embodiments, the peptide-tagged PEGylated chitosan polymer prepared, by method 1 is a scaled-up procedure. This scale-up procedure was successfully accomplished and yielded 60-70% of the final product with enhanced quality as determined and analyzed by FTIR analysis (see FIG. 2). The intensity of peaks obtained vis serial synthetic steps of modifications were high, confirming a better quality product.

This polymer was used to make nanoparticles by complexing siRNA. The polymer easily dissolved in 0.5% acetic acid solution (without healing or sonication), whereas a usual unmodified chitosan polymer only dissolves in 1 to 2% acetic acid solution. The ability to dissolve at lower acetic acid concentration confirms the successful deprotection of the amine groups on the polymer using hydrazine monohydrate. The polymer after dissolution in 0.5% acetic acid solution formed nanoparticles with siRNA and showed higher complexation ability.

We had earlier reported a complexation of 2 μg of siRNA/0.5 mg of polymer (Malhotra et al, 2013a and 2013b) and 8 μg of siRNA/0.5 mg of polymer (Malhotra et al 2013c) using a low molecular weight chitosan as a parent polymer. However, this scale-up procedure led to the binding of 12 μg of siRNA/0.5 mg of polymer (using low molecular weight chitosan as a parent polymer).

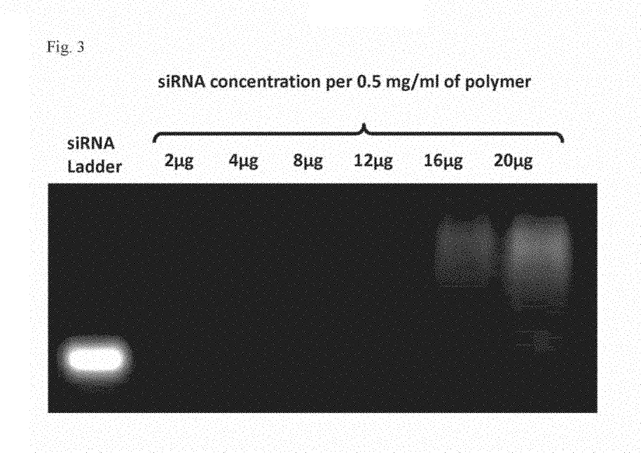

This enhanced encapsulation efficiency was continued by gel retardation assay (FIG. 3). This is an added advantage as it leads higher encapsulation of the payload (dosage form) for the same amount of polymer, in alternative embodiments, when a TAT peptide is used, this improvement in the siRNA binding efficiency is because of the additional amines present on the TAT peptide (rich in arginines), conjugated to the PEGylated chitosan polymer.

In addition, the particle size and surface charge as analyzed by the zetasizer confirmed the size of particles in the range of 200-250 nm with a positive 15-20 mV of surface charge. This result is similar to what we have obtained previously.

Thus, the exemplary methods as provided herein have been confirmed to be successful scale-ups of the synthesized CS-PEG-TAT polymer, which in alternative embodiments can also have enhanced siRNA binding efficiency without compromising on the particle size and surface charge characteristics.

The second exemplary method as provided herein is a variation of the first exemplary method. This method involves the use of homo-bifunctional PEG instead of the monofunctional PEG used in the first method. This alternative exemplary procedure eliminates the use of ethanethiol reagent, which is a harsh and a stinky chemical and causes much inconvenience to use a higher scale due to its odor. In alternative embodiment, one advantage of eliminating the ethanethiol step is that it leads to the reduction of synthesis route from a total of 7 steps to 5 steps.

References: (a) Malhotra M, Tomaro-Duchesneau C and Prakash S (2013a) Synthesis of TAT peptide tagged PEGylated chitosan nanoparticles tor siRNA delivery targeting neurodegenerative diseases, Biomaterials Vol. 34, 1270-1280.

(b) Malhotra M, Tomaro-Duchesneau C, Saha S and Prakash S (2013b), Intranasal siRNA delivery to the brain by TAT/MGF tagged PEGylated chitosan nanoparticles. Journal of Pharmaceutics, Vol. 2013. Article ID: 812187, 10 pages

(c) Malhotra M, Tomaro-Duchesneau C, Saba S and Prakash S (2013c), Systemic siRNA delivery via peptide tagged polymeric nanoparticles, targeting PLK1, gene in a mouse xenograft model of colorectal, cancer. International Journal of Biomaterials, Vol. 2013, Article ID: 252531, 13 pages.

The following examples are provided to further illustrate the invention but not to limit its scope. Other variants of the invention will be readily apparent to one of ordinary skill in the art.

### EXAMPLES

Standard procedures and chemical transformation and related methods are well known to one skilled in the art, and such methods and procedures have been described, for example, in standard references such Fiesers' Reagents for Organic Synthesis, John Wiley and Sons, New York, N.Y., 2002; Organic Reactions, vols. 1-83, John Wiley and Sons, New York, N.Y., 2000, March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All tests and references, patents and patent applications cited herein are expressly incorporated by reference their entirety.

Reactions using compounds having functional groups may be performed on compounds with functional groups that may be protected. A “protected” compound or derivatives means derivatives of a compound where one of more reactive site or sites or functional groups are blocked with protecting groups. Protected derivatives are useful in the preparation of the compounds of the present invention or in themselves; the protected derivatives may be the biologically active agent. Examples suitable protecting groups that can be used to practice this invention can be found in e.g., T. W. Green, Protecting Groups in Organic Synthesis, 3rd edition, John Wiley & Sons, Inc. 1999; or T. W. Green and P. G. M. Wuts, Protecting Groups in Organic Synthesis, 4th edition, John Wiley & Sons, Inc. 2007.

### Example 1—Exemplary “Scale-Up Protocol” for Preparation of Peptide-Tagged PEGylated Chitosan Polymer

In alternative embodiments, this example describes an exemplary “scale-up protocol” for preparation of peptide-tagged PEGylated chitosan polymer.

### Scale-Up Protocol

1. Preparation of phthaloyl chitosan (CSPH) (protecting amine groups on chitosan): 10 grams of commercially available chitosan (range of molecular weights) is added to a solution of Phthalic anhydride (13.8 grams) in 200 ml (or 400 ml) of N,N-Dimethyl Formamide (DMF). The use of phthalic anhydride is 3 mole excess of chitosan used. The mixture is stirred for 8 (to 10 hrs) under nitrogen atmosphere at 110° C. The resultant product is then cooled to room temperature (RT) and precipitated in ice-cold water and filtered. The filtered product is then washed with methanol overnight (12-16 hrs) and vacuum dried. The yield of the product is 200%.
2. Preparation of Carboxyl terminated PEG-monomethyl ether (mPEG-COOH): 60 grams of PEG-monomethyl ether (range of molecular weights) is dissolved in 200 ml (or 400 ml) of Toluene (Range: 10%-30% v/v PEG solution) at 60° C. under nitrogen atmosphere 12 grams of succinic anhydride is dissolved in 50 ml (or 100 ml) of pyridine and added to the PEG solution drop wise. The use of succinic anhydride should be 4 mole excess of PEG. The reaction is then stirred and refluxed (100° C.-110° C.) for 6 hrs (to 12 hrs). The product is cooled to RT and is precipitated with 2 L of ethyl ether, filtered and again re-precipitated with chloroform (100 ml) and diethyl ether (2 L) and vacuum dried. The yield of the product is >95%.
3. Preparation of PEG-grafted phthaloyl chitosan polymer (CSPH-O-mPEG): Prior to this step. 11 gram of PHCS (step 1) is pre-dissolved in 200 ml of Pyridine and stirred overnight (12 to 16 hrs) at room temperature (RT). 330 grams of mPEG-COOH (step 2) is dissolved in 600 ml of toluene at 60° C. under nitrogen conditions. After complete dissolution of mPEG-COOH in toluene, equimolar (or 2 fold molar excess) of Thionyl chloride (SOCl₂) is added to the reaction. The reaction is stirred for 6 hrs (or 8 hrs) and refluxed to boil, followed by degassing to remove excess SO₂ and thioyl chloride. This is an intermediate product that activates mPEG-COOH to mPEG-COCl.

After the mPEG-COCl generating reaction is complete, it is brought to RT and the pre-dissolved PHCS solution in pyridine (200 ml) is added drop-wise to the mPEG-COCl and the reaction, is stirred at RT for 2 hrs under nitrogen, atmosphere, followed by 24 hrs of stirring at 100° C. (or refluxed to boil). After the reaction is complete, the product is precipitated in methanol and vacuum dried to obtain mPEG-PHCS. The yield of the product is >100%.
4.Preparation of Hydroxyl terminated PEGylated Phthaloyl Chitosan (CSPH-O-PEG-OH): 13 grams of CSPH-O-mPEG (step 3) is mixed with 756 mg of Aluminum Chloride (AlCl₃) in 260 ml of ethanethiol and the reaction is stirred at RT for 12 hrs (to 24 hrs). The use of Aluminum Chloride (AlCl₃) is equimoles to the polymer. The reaction mix is diluted with 200 ml of water and acidified with 10% HCl (50 ml) is added. The resulting nurture is then filtered and the product is extracted thrice in dichloromethane. The yield of the product is 90-100%.
5.Preparation of Carboxyl terminated PEGylated Phthaloyl Chitosan (CSPH-O-PEG-COOH): 10 grams of CSPH-O-PEG-OH (step 4) is mixed with 400 ml of Toluene and 1.74 grams of succinic anhydride, pre-dissolved in 50 ml of pyridine is added drop wise to it. The use of succinic anhydride is 4 mole excess of the polymer (CSPH-O-PEG-OH). The reaction is stirred at 100° C. for 12 hrs under nitrogen atmosphere. The reaction is then brought to RT and is precipitated in methanol and vacuum dried. The yield of the product will be >95%.
6.Conjugating TAT peptide to PEGylated Phthaloyl Chitosan (CSPH-O-PEG-TAT): 5 grams of CSPH-O-PEG-COOH (step 5) is dissolved in 250 ml of DMF at 60° C. for 2 hrs. To this, 543 mg of EDC, pre-dissolved in 25 ml of DMF is added dropwise. 2.92 grams of TAT peptide (Mol. wt: 1338.65) pre-dissolved in 20 ml of DMF is added to the reaction mix dropwise and finally 26 mg of DMAP pre-dissolved in 5 ml of DMF is added to the reaction mix, dropwise. The reaction is then continued to stir at 40° C. (range: 25° C. to 60° C.) for 24 hrs. The reaction is than brought to RT and precipitated in water and filtered. Any peptide (for example, a cell targeting/cell penetrating peptide) that has amine at both its terminal group can be used. The filtered product is then washed in methanol and again filter dried. The yield of the resultant product is 90%.
7. Removal of phthaloyl group (deprotection) from TAT peptide-tagged PEGylated phthaloyl chitosan (CS-O-PEG-TAT) 5 gram of CS-PH-O-PEG-TAT (step 6) is dissolved in 100 (to 150 ml) of DMF at 80° C. (to 100° C.) and 13 ml of hydrazine monohydrate is added dropwise to the polymer solution. The reaction is stirred at 80° C. (to 100° C.) for 2 hrs under nitrogen atmosphere. For the reaction to succeed to completion the ratio of HMH to DMF used is 1:7.6 v/v. The resultant reaction is brought to room temperature and precipitated in ethanol and washed thoroughly in ethanol (12-16 hrs), vacuum filtered and air dried. The yield of the resultant product is 60-70%.

The polymer obtained via this scaled-up synthesis method was soluble in 0.5% acetic acid. Showed 6 times higher siRNA binding efficiency than what we have previously observed and reported. Formed nanoparticles in the range of 200-250 nm with a surface charge (zeta potential) of 15-20 mV.

### Example 2—Exemplary “Scale-Up Protocol” for Preparation of Phthaloyl Chitosan

In alternative embodiments, this example describes an exemplary “scale-up protocol”for preparation of peptide-tagged PEGylated chitosan polymer.

In alternative embodiments, provided is an exemplary protocol for the synthesis of Chitosan-PEG-peptide polymer to form a nanoparticle for gene delivery. This exemplary protocol, a variation of the exemplary protocol of Example 1, avoids use of Ethanethiol in step 4, which is a strong and stinky chemical reagent and causes much inconvenience to use because of its odor. Ethanethiol is essentially used to remove the methyl ether group from the mono functional PEG (OH-PEG-monomethyl ether).

This exemplary protocol is an alternative to the exemplary protocol of Example 1 to avoid the use of Ethanethiol reagent without compromising on the quality of the polymer end-product to form nanoparticles: it utilizes homo-functional PEG terminated with hydroxyl groups on both the ends.

In addition, with incorporating the use of homofunctional PEG (OH-PEG-OH) instead of monofunctional PEG (mPEG-OH), steps 4 and 5 of the exemplary synthesis scheme of Example 1 are eliminated. Thus, this alternative exemplary synthesis scheme comes down to a total of 5 synthesis steps instead of 7.
Preparation of phthaloyl chitosan (CSPH) (protecting amine groups on chitosan): 10 grams of commercially available chitosan (range of molecular weights) is added to a solution of Phthalic anhydride (13.8 grants) 200 ml (or 400 ml) of N,N-Dimethyl. Formamide (DMF). The use of phthalic anhydride is 3 mole excess of chitosan used. The mixture is stirred for 8 (to 10 hrs) under nitrogen atmosphere at 110° C. The resultant product is then cooled to room temperature (RT) and precipitated in ice-cold water and filtered. The filtered product then washed with methanol overnight (12-16 hrs) and vacuum dried. The yield of the product will be 200%.
2. Preparation of Carboxyl terminated PEG (COOH-PEG-COOH): 60 grams of OH-PEG-OH (range of molecular weights) is dissolved in 200 ml (or 400 ml) of Toluene (Range: 10%-30% w/v PEG solution) at 60° C. under nitrogen atmosphere. 12 grams of succinic anhydride is dissolved in 50 ml (or 100 ml) of pyridine and added to the PEG solution drop wise. The use of succinic anhydride should be 4 mole excess of PEG. The reaction then stirred and refluxed (100° C.-110° C.) for 6 hrs (to 12 hrs). The product is cooled to RT and is precipitated with 2 L of ethyl ether, filtered and again re-precipitated with chloroform (100 ml) and diethyl ether (2 L) and vacuum dried The yield of the product will be >95%.
3. Preparation of PEG-grafted phthaloyl chitosan polymer (CSPH-O-PEG-COOH: Prior to this step, 11 gram of PHCS (step 1) is pre-dissolved in 200 ml of Pyridine and stirred overnight (12 to 16 hrs) at about RT. 300 grams of COOH-PEG-COOH (step 2) is dissolved in 600 ml of toluene at 60° C. under nitrogen conditions. After complete dissolution COOH-PEG-COOH in toluene, equimolar (or 2 fold molar excess) of thionyl chloride (SOCl₂) is added to the reaction. The reaction is stirred for 6 hrs (or 8 hrs) and refluxed to boil, followed by degassing to remove excess SO₂ and thioyl chloride. This is an intermediate product that activates COOH-PEG-COOH to COCl-PEG-COCl.

After the reaction is complete, it is brought to RT and the pre-dissolved PHCS solution in pyridine (200 ml) is added drop-wise to the COCl PEG-COCl and the reaction is stirred at RT for 2 hrs under nitrogen atmosphere, followed by 24 hrs of stirring at 100° C. (or refluxed to boil). After the reaction is complete, the product is precipitated in water and vacuum dried to obtain COOH-PEG-PHCS. The yield of the product will be >100%.

In this step the precipitation is performed in water unlike in methanol, as performed in the exemplary synthesis scheme of Example 1. The precipitation in water will convert the unreacted COCl groups on the polymer (chitosan-PEG-COCl) back to COOH (chitosan-PEG-COOH). The unreached excess PEG will dissolve in water and the desired product will precipitate out. For this reaction to succeed PEG used should be 5 to 10 times mole excess of phthaloyl chitosan.
4.Conjugating TAT peptide to PEGylated Phthaloyl Chitosan (CSPH-O-PEG-TAT): 5 grams of CSPH-O-PEG-COOH (step 3) is dissolved in 250 ml of DMF at 60° C. for 2 hrs. To this, 543 mg of EDC, pre-dissolved in 25 ml of DMF is added dropwise. 2.92 grams of TAT peptide (Mol. wt. 1338.65) pre-dissolved in 20 ml of DMF is added to the reaction mix dropwise and finally 26 mg of DMAP pre-dissolved in 5 ml of DMF is added to the reaction mix, dropwise. The reaction is then continued to stir at 40° C. (range: 25° C. to 60° C.) for 24 hrs. The reaction is then brought to RT and precipitated in water and filtered. Any peptide (e.g., a cell targeting/cell penetrating peptide) that has amine at both its terminal group can be used. The filtered product is then washed in methanol and again filter dried. The yield of the resultant product will be 90%.
5.Removal of phthaloyl group from TAT peptide tagged PEGylated phthaloyl chitosan (CS-o-PEG-TAT): 5 gram of CS-PH-O-PEG-TAT (step 4) is dissolved in 100 (to 150 ml): of DMF at 80° C. (to 100° C.) and 13 ml of hydrazine monohydrate (HMH) is added dropwise to the polymer solution. The reaction is stirred at 80° C. (to 100° C.) for 2 hrs under nitrogen atmosphere. For the reaction to succeed to completion the ratio of HMH to DMF used is 1:7.6 v/v. The resultant reaction is brought to room temperature and precipitated in ethanol and washed thoroughly in ethanol (12-16 hrs), vacuum filtered and air dried. The yield of the resultant product will be 60-70%.

Although the foregoing invention, has been described in some detail by way of illustration and example for purposes of clarity of understanding, it will, be readily apparent to one of ordinary skill in the art in light of the teachings of this application that certain changes and modifications may be made thereto without departing from the spirit or scope of the appended claims. A number of aspects of the invention have been described. Nevertheless, it will be understood that various modifications may be made without departing from the spirit and scope of the invention. Accordingly, other aspects are within the scope of the following claims.

1. A method or synthetic scheme for the preparation of a phthaloyl chitosan (CSPH) comprising:
(a) providing a chitosan, wherein optionally the chitosan has a range of molecular weight (MW) of between about 10 to 220 KDa, and optionally the chitosan is substantially pure with a deacetylation of between about 75 to 85%
(b) providing a solution of phthalic anhydride, optionally at about 465.90 millimolar (mM), or equivalent, in a solvent comprising an N,N-Dimethyl Formamide (DMF) or equivalent;
(c) mixing a sufficient amount of the chitosan of (a) with the solution of (b) such that the final amount of phthalic anhydride or equivalent is in 3 mole excess of the chitosan;
(d) stirring the mixture of (c) under a nitrogen atmosphere or equivalent non- oxygen atmosphere at a temperature above a 100° C., optionally stirring for between about 6 to 10 hours, or for 6, 7, 8, 9, or 10 hours, and optionally stirring at a temperature of between about 100° C. and 120° C., or at 110° C.;
(e) cooling the stirred mixture first to about room temperature (RT) or between about 21° C. to 24° C., then adding the reaction mix in excess of water at 0° C. to 15° C. (optionally cooling in ice water or ice cold water) to generate a precipitate of phthaloyl chitosan (CSPH);
(f) isolating the precipitate of CSPH, optionally by filtering, and washing the CSPH with a solvent comprising a methanol or equivalent, in excess, optionally washing overnight or between about 12 to 16 hours; and
(g) drying, optionally vacuum drying, the CSPH-comprising solvent to yield a CSPH product.

2. A method or synthetic scheme for the preparation of a carboxyl terminated PEG-monomethyl ether (mPEG-COOH), comprising:
(a) providing a polyethylene glycol (PEG)-monomethyl ether (PEG-MME), wherein optionally the PEG-MME has a range of molecular weight (MW) of between about 2000 Da to 10,000 Da, and optionally the PEG-MME is substantially pure;
(b) providing a solvent comprising a toluene or equivalent;
(c) mixing the PEG-MME with the solvent of (b) to a range of between about 10% to 30% PEG-MME, under a nitrogen atmosphere or equivalent non-oxygen atmosphere at between about 50° C. and 70° C., or at 60° C.;
(d) providing a solution of succinic anhydride in pyridine at concentration equivalent to about 12 grams (gms) succinic anhydride dissolved in between about 50 ml to 100 ml pyridine;
(e) adding the solution of (d) dropwise or incrementally to the PEG-MME solution of step (c) until succinic anhydride is at about a 4-fold mole excess of the PEG-MME;
(e) stirring with refluxing the final solution of (e) at a temperature above
100° C., or at a temperature between about 100° C. to 120° C., or 110° C., optionally for between about 5 to 15 hours, or 6 to 12 hours;
(f) cooling the stirred solution of (e) to about room temperature (RT) or between about 21° C. to 24° C., and precipitated with a solvent comprising an ethyl ether (EE), optionally using about 2 liters (L) EE or equivalent;
(g) isolating the precipitate of (f), optionally by filtering, and re-dissolving the precipitate by adding a solvent comprising a chloroform or equivalent and again re-precipitating in diethyl ether or equivalent, optionally the solvent comprising the equivalent of 100 ml chloroform or equivalent and 2 L diethyl ether or equivalent; and
(h) drying, optionally vacuum drying, the solvent of (g) to yield a dry carboxyl terminated PEG-monomethyl ether (mPEG-COOH) product.

3. A method or synthetic scheme for the preparation of a PEG-grafted phthaloyl chitosan polymer (CSPH-O-mPEG), comprising:
(a) providing a phthaloyl chitosan (CSPH), optionally a phthaloyl chitosan (CSPH) made by the method of claim 1, and dissolving the CSPH in a solvent comprising a pyridine or equivalent, stirring (optionally stirring overnight, or between about 5 to 20 hours or 12 to 16 hours) at about room temperature (RT) or at between about 21° C. to 24° C.;
(b) providing a carboxyl terminated PEG-monomethyl ether (mPEG-COOH), or the carboxyl terminated PEG-monomethyl ether (mPEG-COOH) product of claim 2, and dissolving in a solvent comprising a toluene or equivalent at between about 50° C. and 70° C., or about 60° C., under a nitrogen atmosphere or equivalent non-oxygen atmosphere, and after complete dissolution in the solvent add a thionyl chloride (SOCl₂) at an amount of between about equimolar to 2-fold molar excess;
(c) stirring and refluxing under boiling conditions the final solution of (b) for between about 5 to 10 hours (hrs) or 6 to 8 hrs, followed by degassing to remove excess SO₂ and thioyl chloride, to generate a mPEG-COCl product;
(d) lowering the temperature of the mPEG-COCl product-comprising solution of (c) to about room temperature (RT) or at between about 21° C. to 24° C., and adding dropwise or incrementally the CSPH solution of step (a), optionally adding about 200 ml, and stirring at about room temperature (RT) or at between about 21° C. to 24° C., for between about 1 to 5 hrs or about 2 hrs under a nitrogen atmosphere or equivalent non-oxygen atmosphere;
(e) stirring the final solution of (d) for between about 18 hrs to 30 hrs or about 24 hrs under boiling conditions or conditions comprising boiling and refluxing; and
(f) precipitating a CSPH-O-mPEG product in excess of a solvent comprising a methanol or equivalent, thereby generating a CSPH-O-mPEG product, and optionally drying (optionally vacuum drying) to generate a dry mPEG-PHCS or CSPH-O-mPEG product.

4-8. (canceled)

9. A peptide tagged PEGylated chitosan (CS-O-PEG-peptide) made by the methods or synthetic schemes of claim 1,
wherein optionally the CS-O-PEG-peptide is: (a) soluble in 0.5% to 1% acetic acid; (b) has 6 times higher siRNA binding efficiency than a CS-O-PEG-peptide of less purity; (c) formed nanoparticles in the range of between about 200 nm to 250 nm with a surface charge (zeta potential) of 15-20 mV; or (d) any combination of (a), (b) and (c),
and optionally the peptide tagged PEGylated chitosan is CS-O-PEG-TAT if the peptide is a TAT,
and optionally the peptide tagged PEGylated chitosan is about 60 to 100% pure.

10-16. (canceled)
